(19) United States
(12) Patent Application Publication
(10) Pub. No.: US 2008/0281361 A1
(43) Pub. Date: Nov. 13, 2008

(54) Posterior stabilization and spinous process systems and methods

(76) Inventors: Shannon Marlece Vittur, Memphis, TN (US); Aurelian Bruneau, Memphis, TN (US); Eric C. Lange, Collierville, TN (US); Kent M. Anderson, Memphis, TN (US)

Correspondence Address: COATS & BENNETT/MEDTRONIC, 1400 CRESCENT GREEN, SUITE 300, CARY, NC, 27518, US

(21) Appl. No.: 11/801,603

(22) Filed: May 10, 2007

(51) Int. Cl. A61B 17/70; A61B 17/56; A61B 17/58; A61F 2/44

(52) U.S. Cl. 606/249; 606/100; 623/17.16; 606/246; 606/250; 606/278

(57) ABSTRACT

Posterior spinal stabilization devices and methods include first and second elongate elements engageable along the spinal column and a spinous process replacement body positionable between the elongate elements. Connection mechanisms are provided to adjustably connect the spinous process replacement device to the elongate elements so that the spinous process replacement device can be moved to the desired location between the connecting elements and secured in the desired location.

### BACKGROUND

Spinal stabilization procedures are performed and include placement of devices between vertebral bodies in the disc space or along the spinal column. For example, varieties of inter-body fusion devices are widely used following partial or total discectomies to fuse adjacent vertebrae. Artificial disc devices can be placed in the disc space if motion preservation is desired. Still other stabilization devices contemplate the attachment of plates, rods or tethers extradiscally along the vertebrae. Still others are positioned between spinous processes.

In some procedures, the spinous process of the patient may be damaged or otherwise compromised such that it is not capable of supporting an interspinous stabilization element in a stabilization procedure. In other procedures, the spinous process is removed. There remains a need for devices for spinal stabilization procedures that replace the spinous process and can be integrated with posterior stabilization instrumentation so that interspinous stabilization procedures can be completed even if the spinous process of the patient is removed as a result of being compromised or to accommodate the surgical procedure.

### SUMMARY

Posterior spinal stabilization devices and methods include first and second elongate elements engageable along the spinal column and a spinous process replacement body positionable between the elongate elements. Connection means are provided to adjustably connect the spinous process replacement device to the elongate elements so that the spinous process replacement device can be moved to the desired location between the connecting elements and secured in the desired location.

According to one aspect, a posterior spinal stabilization system comprises first and second elongate elements and a replacement body positionable between the first and second elongate elements. The replacement body includes a spinous process portion alignable along a vertebral body to replace a spinous process of the vertebral body. The system also includes connection means for adjustably engaging the replacement body and the first and second elongate elements to one another. The connection means is structured to permit the replacement body between the first and second elongate elements to be adjusted by moving the replacement body along the connection means transversely to the first and second elongate elements.

According to another aspect, a posterior spinal stabilization system comprises first and second elongate elements and a replacement body positionable between the first and second elongate elements. The replacement body includes a spinous process portion alignable along a vertebral body to replace a spinous process of the vertebral body. The system also includes first and second linking arms engaged to respective ones of the first and second elongate elements. The replacement body is adjustably connected to the first and second linking arms and is movable along the linking arms to a desired location between the first and second elongate elements.

According to another aspect, a method for posterior spinal stabilization comprises: engaging first and second elongate elements posteriorly along the spinal column, the first and second elongate elements being located on opposite sides of the sagittal plane; positioning a spinous process replacement body between the first and second elongate elements adjacent a vertebra of the spinal column; securing first and second linking arms to respective ones of the first and second elongate elements; adjusting a position of the spinous process replacement body along the vertebra in the medial-lateral direction; and engaging the spinous process replacement body to the first and second linking arms to fix the spinous process replacement body in the position.

These and other aspects will be discussed further below.

### BRIEF DESCRIPTION OF THE DRAWINGS

FIG. 1 is a schematic view of a posterior spinal stabilization system and a spinal column segment.

FIG. 2 is a perspective view of one embodiment of a posterior spinal stabilization system.

FIG. 3 is a perspective view of a spinous process replacement body of the stabilization system of FIG. 2.

FIG. 4 is a side elevation view of another embodiment replacement body.

FIG. 5 is perspective view of a coupling member of the stabilization system of FIG. 2.

FIG. 6 is a perspective view of a locking member of the stabilization system of FIG. 2.

FIG. 7 is a perspective view of a linking arm of the stabilization system of FIG. 2.

FIG. 8 is a perspective view of another embodiment linking arm of the stabilization system of FIG. 2.

FIG. 9 is a perspective view of a securing member of the stabilization system of FIG. 2.

FIG. 10 is a side elevation view showing the linking arm of FIG. 7 engaged to the replacement body to provide a mechanical stop to prevent or limit the replacement body from rotating relative to the linking arm.

FIG. 11 is a side elevation view showing another embodiment rotational stop arrangement to prevent or limit the spinous process replacement body from rotating relative to the linking arm.

FIG. 12 is a side elevation view in partial section showing another embodiment rotational stop arrangement to prevent or limit the spinous process replacement body from rotating relative to the linking arm.

FIG. 13 is a side elevation view in partial section showing another embodiment rotational stop arrangement to prevent or limit the spinous process replacement body from rotating relative to the linking arm.

FIG. 14 is a perspective view of another embodiment posterior stabilization system including a spinous process replacement body of FIG. 4 therein.

FIG. 15 is another perspective view of the posterior stabilization system of FIG. 14.

FIG. 16 is a perspective view of another embodiment posterior stabilization system with a spinous process replacement body.

FIG. 17 is a perspective view of another embodiment posterior stabilization system with a spinous process replacement body.

FIG. 18 is a perspective view of another embodiment posterior stabilization system with a spinous process replacement body.

FIG. 19 is a perspective view of another embodiment posterior stabilization system with a spinous process replacement body.

FIG. 20 is a perspective view of another embodiment posterior stabilization system with a spinous process replacement body.

FIG. 21 is a perspective view of another embodiment posterior stabilization system with a spinous process replacement body.

FIG. 22 is a perspective view of another embodiment posterior stabilization system with a spinous process replacement body.

FIG. 23 is a perspective view of another embodiment posterior stabilization system with a spinous process replacement body.

FIG. 23A is a section view along line 23A-23A of FIG. 23.

FIG. 24 is a perspective view of another embodiment posterior stabilization system with a spinous process replacement body.

FIG. 25 is a perspective view of another embodiment posterior stabilization system with a spinous process replacement body.

FIG. 26 is a perspective view of another embodiment posterior stabilization system with a spinous process replacement body.

### DESCRIPTION OF THE ILLUSTRATED EMBODIMENTS

For the purposes of promoting an understanding of the principles of the invention, reference will now be made to the embodiments illustrated in the drawings and specific language will be used to describe the same. It will nevertheless be understood that no limitation of the scope of the invention is thereby intended, such alterations and further modifications in the illustrated devices, and such further applications of the principles of the invention as illustrated therein being contemplated as would normally occur to one skilled in the art to which the invention relates.

Posterior stabilization systems include a spinous process replacement body implantable adjacent the spinal column to replace a spinous process of a vertebra. The spinous process replacement body can also reinforce or replace all or a portion of a lamina resected, removed, or altered along with the spinous process. The spinous process replacement body includes a spinous process portion implantable along a vertebra adjacent the posterior elements of the vertebra to replace a removed or compromised spinous process. The stabilization systems also include connection means for connecting the replacement body between elongate elements extending along the spinal column that are positioned bi-laterally of the posterior midline or sagittal plane of the spinal column. The connection means are adjustable to allow the location of the replacement body between the elongate elements to be readily adjusted.

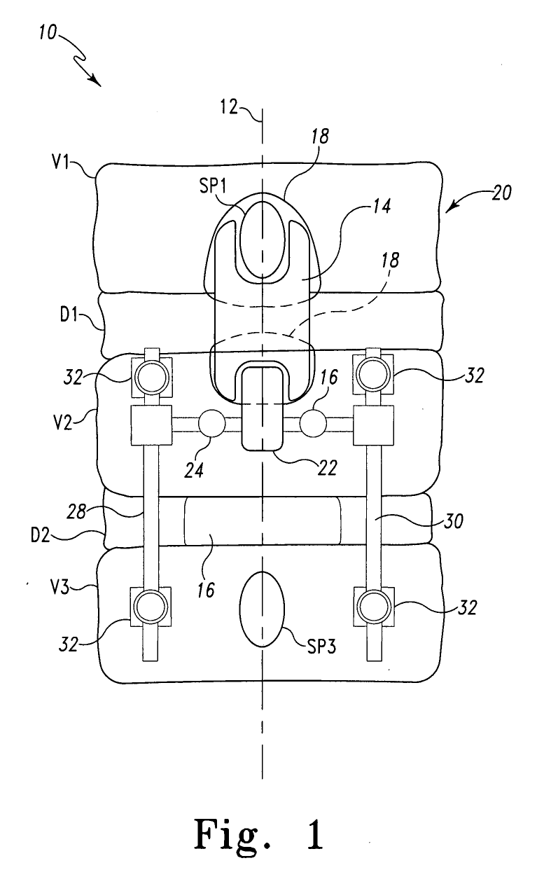

In FIG. 1 there is shown a schematic of a posterior stabilization system 20 and a posterior elevation view of a spinal column segment 10. Spinal column segment 10 includes a number of vertebrae V1, V2, V3 extending along a central axis 12 of spinal column segment 10. Stabilization system 20 includes a spinous process replacement body 22 and opposite connection mechanisms 24, 26 extending medially-laterally from replacement body 22. First and second elongate elements 28, 30 extend along spinal column segment 10 in the direction of central axis 12. Elongate elements 28, 30 are locate bi-laterally along vertebrae V2, V3 on opposite sides of the sagittal plane, and are secured to one or more of the vertebrae with anchors 32. Connection mechanisms 24, 26 are engageable to respective ones of the elongate elements 28, 30 and replacement body 22 to secure replacement body 22 adjacent one of the vertebrae V along its posterior side. At least one of the connection mechanisms 24, 26 includes adjustment means to allow the positioning of replacement body 22 to be adjusted medially-laterally between elongate elements 28, 30 and transversely to the sagittal plane and central axis 12 to the desired alignment with vertebra V2.

In FIG. 1 replacement body 22 is positioned along the posterior side of vertebra V2 and is aligned generally along central axis 12 posteriorly of the spinal canal to replace a spinous process removed from vertebra V2. Vertebra V1 includes a spinous process SP1 aligned generally along central axis 12. Interspinous implant 14 is positioned between replacement body 22 and spinous process SP1 to provide support of the vertebrae V1, V2. In one embodiment, interspinous implant 14 is flexible to permit motion of the vertebrae V1, V2 in extension and rotation. One or more tethers 18 or other devices can be secured between interspinous implant 12 and one or both of spinous process SP1 and replacement body 22 to secure interspinous implant 14 thereto. Tethers 18 may also be engaged to the lamina or to other structure of the vertebrae V1, V2.

Interspinous implant 14 is abuttingly engaged with spinous process SP1 and replacement body 22 to provide and maintain separation between spinous process SP1 and replacement body 22. In one embodiment, interspinous implant 14 is the DIAM™ Spinal Stabilization System of Medtronic, Inc. employed for spinal decompression surgery between spinous processes of the patient to alleviate spinal stenosis. Interspinous implant 14 is placed between the spinous process SP1 and replacement body 22 to act as a shock absorber and reduce loads on the surrounding vertebrae and restore the natural function of the joint. In one embodiment, the core of interspinous implant 14 is made of silicone or other compressible, resilient material, and the core is surrounded with an outer mesh Interspinous implant 14 includes opposite generally U-shaped ends to fit around the respective spinous process and replacement body 22. One of the U-shaped ends receives the spinous process SP1 therein and the other of the U-shaped ends receives the replacement body 22 therein. The U-shaped ends cradle the replacement body 22 and spinous process SP1 to maintain the interspinous implant 14 in position when positioned therebetween.

Other procedures contemplate that the interspinous implant 14 is made from a rigid material, or includes any other suitable form for positioning between spinous processes and replacement body 22. Examples of suitable materials include titanium, stainless steel, other metals and metal alloys, and non-metal materials including PEEK, polymers, ceramics, and carbon fiber, for example. In one form interspinous implant 14 is a plate, clamp or other device that is engaged to replacement body 22 and one or more other spinous processes of the patient or other replacement bodies. In yet other embodiments, multi-level stabilization procedures are contemplated by positioning a second interspinous implant 14 between replacement body 22 and spinous process SP3. In another embodiment, a single level stabilization procedure is contemplated by positioning a single interspinous implant 14 between replacement body 22 and spinous process SP3. In still another procedure, an interspinous implant 14 is positioned between two artificial replacement bodies 22 engaged to respective ones of first and second vertebrae. Multi-level stabilization procedures with interspinous implants and spinous process replacement bodies are also contemplated.

Elongate elements 28, 30 extend along vertebrae V2, V3, and are secured to vertebrae V2 and V3 with anchors 32. Elongate elements 28, 30 are spinal rods in one embodiment. Other embodiments contemplate other forms for elongate elements 28, 30, including plates, tethers, staples, wires, cables, and other devices. In still other embodiments, the elongate elements 28, 30 have a length sized to extend along more than two vertebrae. In still other embodiments, elongate elements 28, 30 do not have the same length.

Anchors 32 can be any suitable anchor for securing elongate elements 28, 30 to the vertebrae. Anchors 32 can be multi-axial or uni-axial screws, hooks, clamps, interbody devices, bolts, or other device engageable with the respective vertebrae and elongate element. The anchor can be engaged to the pedicle, facet, transverse processes, anterior portion of the vertebral body, or one or more endplates of the respective adjacent vertebrae. It is also contemplated that the anchors securing elongate element 28 need not be engaged to the same vertebrae as the anchors securing elongate element 30.

Stabilization system 20 includes, in one embodiment, at least one interbody device 18 in a disc space between vertebrae, such as shown in disc space D2 between vertebrae V2, V3. Interbody device 18 can be a fusion device to facilitate fusion of the supported vertebrae or a motion preserving device to permit motion of the supported vertebrae. In other embodiments, disc space D1 also includes at least one interbody device. In still other embodiments, disc space D1 includes an interbody device 18 therein supporting vertebrae V1, V2 whereas disc space D2 does not. Interspinous implant 14 can be positioned between the same vertebrae as the interbody device 18, between different vertebrae than the vertebrae between which interbody device 18 is positioned, or in multiple disc spaces along the spinal column segment.

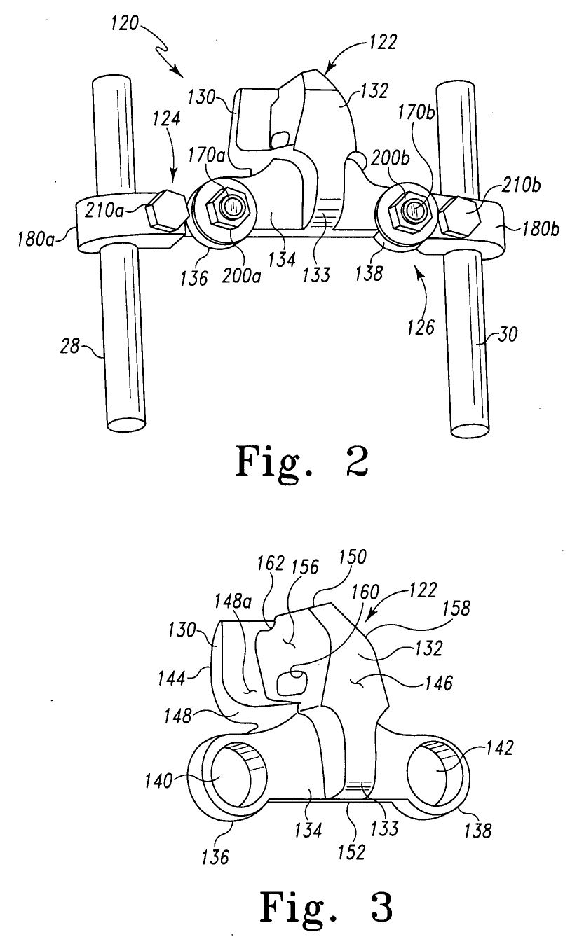

Referring now to FIG. 2, there is shown one embodiment of stabilization system 20 designated as stabilization system 120. Stabilization system 120 includes elongate elements 28, 30, an embodiment of the spinous process replacement body designated at 122, and an embodiment of connection mechanism 24, 26 designated as connection mechanisms 124, 126 extending between replacement body 122 and respective ones of the elongate elements 28, 30. Replacement body 122 is shown in isolation in FIG. 3. Replacement body 122 includes an anterior lamina portion 130 and a posteriorly extending spinous process portion 132 extending from lamina portion 130. A transverse mounting portion 134 extends along the inferior or lower sides of spinous process portion 132 transversely to spinous process portion 132. Mounting portion 134 includes opposite ears 136, 138 that each define a respective hole 140, 142. Replacement body 122 also includes a lower or inferior flange 133 extending along a posterior side of mounting portion 134 and protruding posteriorly therefrom. Replacement body 122 is formed as a single, integral unit. Other embodiments contemplate that replacement body 122 includes multiple components assembled or fastened together to form the replacement body.

Lamina portion 130 is formed by a plate-like body and includes an anterior surface 144 that is oriented toward the spinal canal when implanted and contacts the lamina of vertebra V2 to resist anterior migration of replacement body 122 into the spinal canal. Spinous process portion 132 forms a plate-like posterior extension transversely oriented to lamina portion 130 and extending to a posterior side 146. Spinous process portion 132 also extends between superior and inferior ends 150, 152. The superior end is oriented cephaladly when implanted toward an upper vertebra, and the inferior end is oriented caudally. In one form, lamina portion 130 forms a plate-like portion extending orthogonally to the sagittal plane and spinous process portion 132 forms a plate-like portion that lies within or generally parallel to the sagittal plane. Spinous process portion 132 further includes opposite side surfaces 156, 158 extending between lamina portion 130 and posterior side 146, and further extending between mounting portion 134 and superior end 150. Opposite side surfaces 156, 158 form a width in the direction between elongate elements 28, 30. The width tapers from a maximum width adjacent mounting portion 134 toward superior end 150. The tapered side surfaces facilitate engagement with the interspinous implant 14. In other embodiments, non-tapered configurations for opposite side surfaces 156, 158 are contemplated. The transition between posterior side 146 and superior end 150 is beveled to eliminate or reduce sharp corners or edges therebetween.

Replacement body 122 also includes a pair of identical opposite support members 148 (only one shown) extending from lamina portion 130 and laterally outwardly from and along side surfaces 156, 158 of spinous process portion 128 toward posterior side 146. Support members 148 include a superior support surface 148a to provide a location along which the interspinous implant 14 resides against or is positionable into abutting engagement with when engaged to replacement body 22. Support surfaces 148a are concavely curved and form a smooth transition between lamina portion 130 and the portion of support members 148 along spinous process portion 132 to prevent the formation of sharp, abrupt edges and minimize stress concentrations in the interspinous implant positioned thereagainst.

Spinous process portion 132 also includes a through-hole 160 extending between and opening at the respective side surfaces 156, 158 superiorly of support members 148. Through-hole 160 provides a location through which tethering elements can be positioned to secure interspinous implant 14 to replacement body 122. Through-hole 160 is elongated in the anterior-posterior direction when replacement body 122 is implanted to provide some adjustability in the tether location therethrough. The elongated through-hole can also accommodate tethers formed as flat, wide bands. Other embodiments contemplate other shapes for through-hole 160, including round through-holes, square or rectangular through-holes, and multiple through-holes in side-by-side relation in the anterior to posterior direction. Still other embodiments contemplate a replacement body 122 without a through-hole.

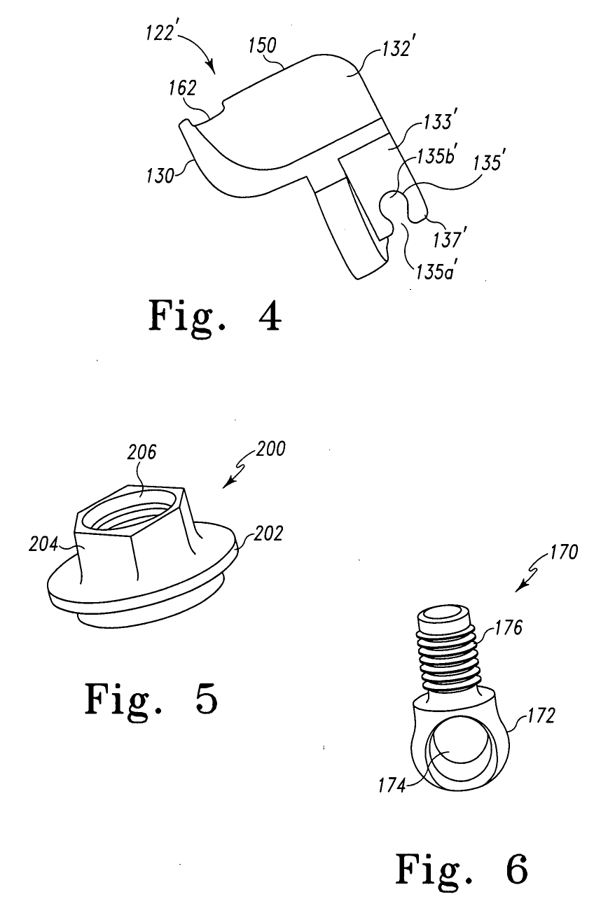

FIG. 4 shows another embodiment replacement body 122′ that is similar to replacement body 122, and like elements are designated with like reference numerals. Replacement body 122′ includes a spinous process portion 132′ that is solid without a through-hole for a tether. Spinous process portion 132′ includes a lower or inferior flange 133′ that has a slotted hole 135′ opening at the inferior end 137′ of flange 133′. Slotted hole 135′ includes a slot portion 135a′ that leads into hole portion 135b′. Tether 18 is side-loaded through slot portion 135a′ and into hole portion 135b′.

Spinous process portion 132 also includes a notch 162 in superior end 150. Notch 162 provides a secure and reliable location in which a distraction instrument can be received to exert distraction forces between the vertebrae V1 and V2 or the vertebrae V2 and V3 through the replacement body 122 and the respect spinous processes SP1, SP3. Notch 162 is formed adjacent the lamina portion 130 so that distraction forces are applied more toward the central axis of the vertebral bodies. Furthermore, lamina portion 130 forms an anterior wall along the notch 162 to prevent the distractor from migrating into the spinal canal during distraction. Notch 162 is U-shaped and longer in the anterior-posterior direction than its depth into spinous process portion 132 to preserve the integrity of spinous process portion 132. Other embodiments contemplate other shapes for notch 162, including V-shapes, semi-circular shapes, and irregular shapes, for example. Still other embodiments contemplate a replacement body 122 without notch 162, or a replacement body 122 with multiple notches.

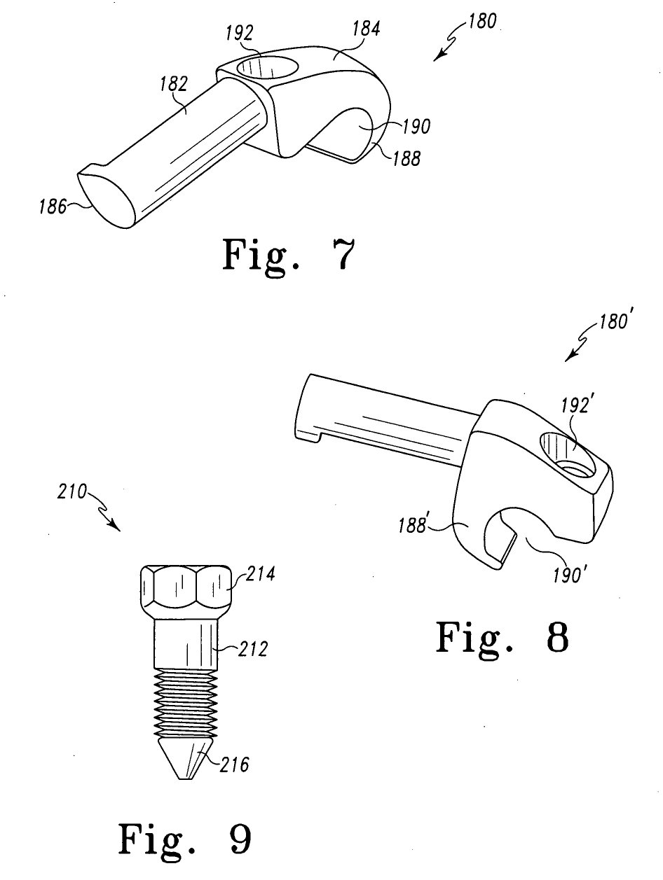

Referring back to FIG. 2, connection mechanisms 124, 126 providing a connection means for adjustably engaging replacement body 122 between elongate elements 28, 30. Connection mechanisms 124, 126 each include a coupling member 170a, 170b (collectively and individually referred to as coupling member 170 and further shown in FIG. 5) that couples respective ones of the linking arms 180a, 180b (collectively and individually referred to as linking arms 180 and shown further in FIG. 7) to mounting portion 134 at respective ones of the ear portions 136, 138. Linking arms 180a, 180b are slidably adjustable in the respective coupling member 170a, 170b so that the location of replacement body 122 along the linking arms 180a, 180b can be adjusted in the medial-lateral direction. Furthermore, coupling members 170 permit the angular orientation of linking arms 180 relative to mounting portion 134 to be readily adjusted to accommodate engagement of linking arms 180 to elongate elements 28, 30 extending in non-parallel arrangements.

Connections mechanisms 124, 126 also include locking members 200a, 200b (collectively and individually referred to as locking members 200 and shown further in FIG. 6) that are engaged to respective ones of the coupling members 170 to lock the corresponding linking arm 180 in position relative to the respective coupling member 170 and mounting portion 134. Linking arms 180 extend from the respective coupling member 170 to the adjacent elongate element 28, 30. Linking arms 180 are secured to the respective elongate element with a respective one of the securing members 210a, 210b (collectively and individually referred to as securing members 210 and further shown in FIG. 9.) Linking arms 180 are adjustable along the respective elongate element 28, 30 to a desired location and locked or secured in position therealong with the respective securing member 210.

Referring to FIG. 5, coupling member 170 includes an eyebolt type arrangement with a receiving portion 172 having a passage 174 extending therethrough, and a stem 176 extending from receiving portion 172. Stem 176 includes an external thread profile extending therealong, and is sized for positioning through holes 140, 142 of mounting portion 134. Stem 176 is sized for rotation in holes 140, 142 to orient the respective linking arm 180 to accommodate elongate elements 28, 30 that converge cephaladly, diverge cephaladly, or extend parallel to one another. Passage 174 is sized to receive a portion of the respective linking arm 180 therein so that the location along the linking arm 180 in which coupling member 170, and thus replacement body 122, is secured can be varied to adjust the location of replacement body 122 medially and laterally between elongate elements 28, 30. The end of stem 176 opposite receiving portion 172 can include an internal recess to receive a tool to manipulate or hold coupling member 170 during assembly.

Once the desired positioning of replacement body 122 relative to vertebral body V2 is obtained, the replacement body 122 is locked in position along the linking arms 180 with locking member 200, as shown in FIG. 6. Locking member 200 includes an end flange 202 and a proximal head portion 204. Head portion 204 includes an outer tool engaging surface such as a hex or any other suitable external or internal shape for engaging a driving or tightening tool. A passage 206 extends through head portion 204 and end flange 202. Passage 206 includes internal threads to threadingly engage stem 176 of the respective coupling member 170. Flange 202 abuts the adjacent component, such as the ear portion 136, 138 of mounting portion 134, to secure it against the respective linking arm 180 and to secure the linking arm 180 in passage 174 against receiving portion 172 of the respective coupling member 170.

Referring now to FIG. 7, there is shown linking arm 180. Linking arm 180 includes an arm portion 182 extending from a linking portion 184. Arm portion 182 includes a circular cross-section along at least a portion of its length, and includes an end portion 186 remote from linking portion 184. Linking portion 184 includes a hook-shaped element 188 that defines a receptacle 190 to receive the respective elongate element 28, 30 therein. Linking portion 184 also includes a bore 192 extending therein to receive securing member 210. Securing member 210 engages the elongate element 28, 30 in receptacle 190 to secure linking arm 180 and the respective elongate element 28, 30 to one another.

In FIG. 7, receptacle 190 is structured so that it opens medially toward the replacement body 122 (as shown in FIG. 2) so that securing member 210 directs the elongate element 28, 30 in receptacle 190 laterally against the inner surface of hook-shaped element 188. In FIG. 8, another embodiment linking arm 180′ is shown that is similar to linking arm 180 except for the orientation of receptacle 190′. Receptacle 190′ opens laterally when implanted and away from replacement body 122, such as shown in FIG. 15. Linking arm 180′ includes hook-shaped element 188′ and bore 192′ to receive securing member 210 to direct the respective elongate element 28, 30 in receptacle 190′ medially into contact with the inner surface of hook-shaped element 188′. In stabilization system 20, it is contemplated that the connection mechanisms 24, 26 employ linking arms that are the same, e.g. two linking arms 180 or two linking arms 180′, or two linking arms that differ from one another.

Referring now to FIG. 9, there is shown securing member 210 that includes a shaft 212 extending between a proximal head 214 and a distal tip 216. Head 214 provides a location for engagement with an inserter instrument. In the illustrated embodiment, head 214 includes an outer hex configuration. Other embodiments contemplate other configurations, including internal hexes, slots, and any suitable driver instrument engagement structure. In still other embodiments, head 214 can be provided with a break-off portion that severs upon application of a threshold torque when securing member 210 is securely engaged to linking arm 180, 180′ and the corresponding elongate element 28, 30.

Shaft 212 includes a threaded portion to threadingly engage bore 190, 190′ of the corresponding linking arm 180, 180′. Other embodiments contemplate other securing arrangements between the securing member and linking arms, including non-threaded arrangements. Tip 216 includes a distally tapered conical profile that resides against the respective elongate element 28, 30 to push it against the inner surface of the hook-shape element of the corresponding linking arm 180, 180′ when engaged thereto. Other embodiments contemplate other arrangements for distal tip 216, including non-tapered arrangements.

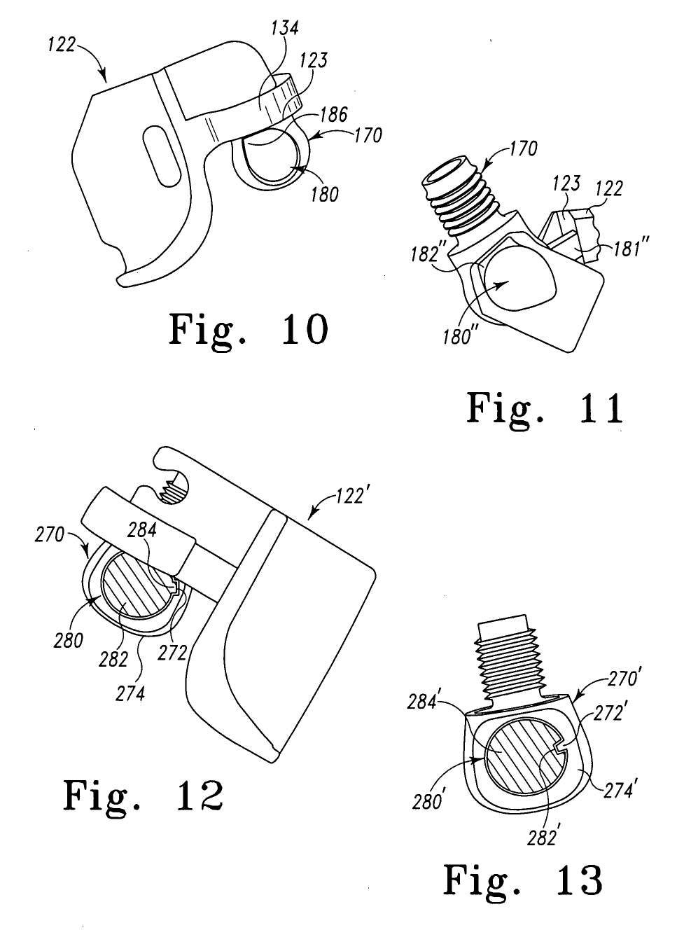

Referring to FIG. 10, there is shown a side view of replacement body 122 engaged to linking arm 180 with coupling member 170. End portion 186 of arm portion 182 of linking arm 180 forms a rotational stop that abuts an anterior surface 123 of mounting portion 134 of replacement body 122 to limit rotation of replacement body 122 about arm portion 182. In FIG. 11, a modified linking arm 180″ is provided with an extension arm 181″ extending from arm portion 182″ that contacts the anterior surface 123 of replacement body 122 to limit or prevent rotation of the replacement body about linking arm 180″.

In FIG. 12, another embodiment linking arm 280 is shown that is similar to linking arm 180, but includes a protrusion 284 extending from arm portion 282 thereof. Coupling member 270 is similar to coupling member 170, but includes a slot 272 in its distal receiving portion 274. Protrusion 284 is received in slot 272 to provide a keyed arrangement that prevents coupling member 270 from rotating relative to linking arm 280, maintaining the coupling member 270 and linking arm 280 in position relative to one another and preventing the replacement body 122′ (or replacement body 122) from rotating to migrate anteriorly toward the spinal canal. In FIG. 13, the keyed arrangement in FIG. 12 is reversed, and the coupling member 270′ includes a protrusion 272′ extending from receiver portion 274′, while linking arm 280′ includes a slot 282′ along its arm portion 284′ that receives protrusion 272′ to resist rotation of the linking arm 280′ and coupling member 270′ relative to one another.

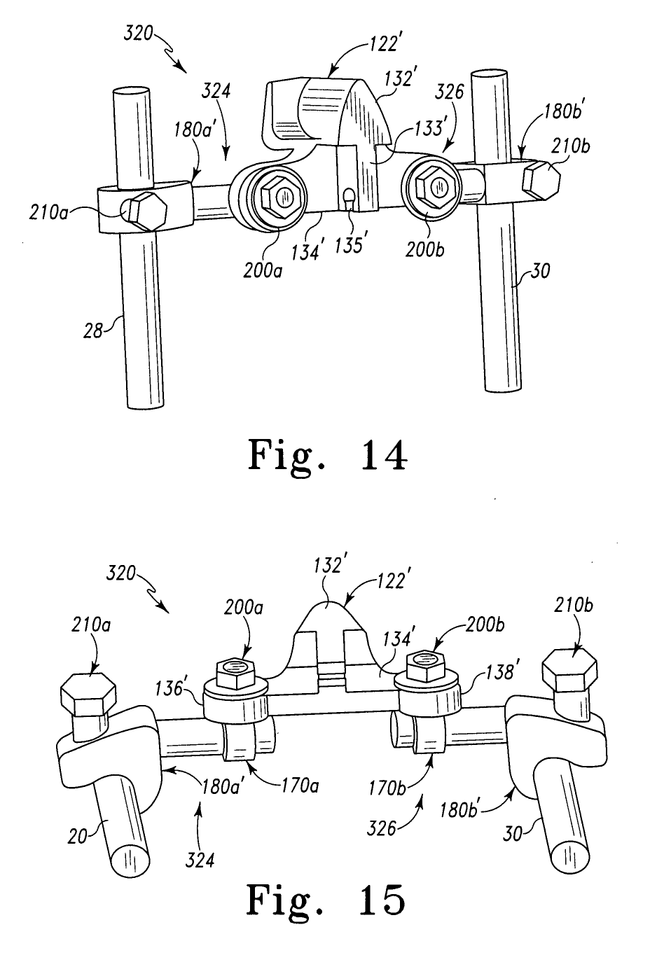

FIGS. 14 and 15 show another embodiment of posterior stabilization system 20 designated as stabilization system 320. Stabilization system 320 includes replacement body 122′ between elongate elements 28, 30. Stabilization system 320 further includes connection mechanisms 324, 326 that adjustably secure replacement body 122′ between elongate elements 28, 30 and in a desired medial-lateral position adjacent a vertebral body. Connection mechanisms 324, 326 include respective ones of linking arms 180a′, 180b′ engaged to elongate elements 28, 30 with the respective securing member 210a. 210b. Linking arms 180a′, 180b′ are coupled to ears 136′, 138′, respectively, of mounting portion 134′ with respective ones of the coupling members 170a, 170b and locking members 200a, 200b in a manner similar to that discussed above with respect to system 120. Slotted hole 135′ is oriented away from spinous process portion 132′ so that a tether extending from an interspinous spacer positioned against spinous process portion 132′ is received in slotted hole 135′ to secure the interspinous spacer to replacement body 122′.

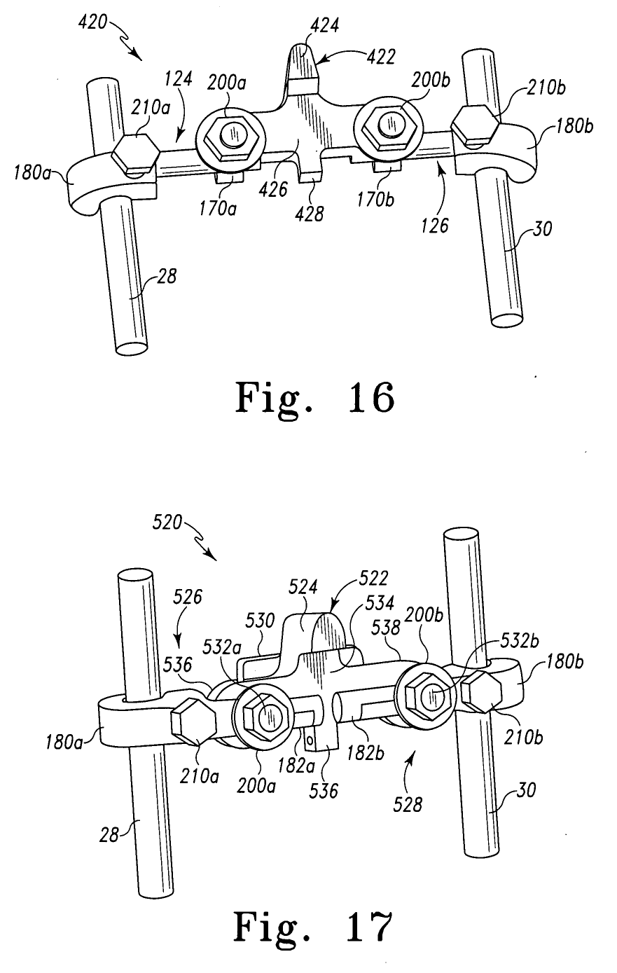

Referring to FIG. 16, there is shown another embodiment of posterior stabilization system 20 designated as stabilization system 420. Stabilization system 420 includes another embodiment replacement body 422 between elongate elements 28, 30. Replacement body 422 includes a spinous process portion 424 extending superiorly from and projecting posteriorly from a central mounting portion 426. Central mounting portion 426 differs from mounting portion 134 in that the posteriorly oriented surface lacks any flange projecting therefrom. Spinous process portion 424 forms a superiorly tapered body to receive the interspinous spacer. Other embodiments contemplate a plate-like portion along the anterior side of spinous process portion 424 to form a lamina portion. An inferiorly extending flange or post 428 extends from mounting portion 426 and oppositely of spinous process portion 424. Mounting portion 426 includes opposite ears with through-holes similar to mounting portion 134 of replacement body 122 as discussed above. First and second connection mechanisms 124, 126 are engaged between respective ones of the ears of mounting portion 426 and the respective elongate element 28, 30 to provide means for medially-laterally adjusting the location of replacement body 422 between elongate elements 28, 30.

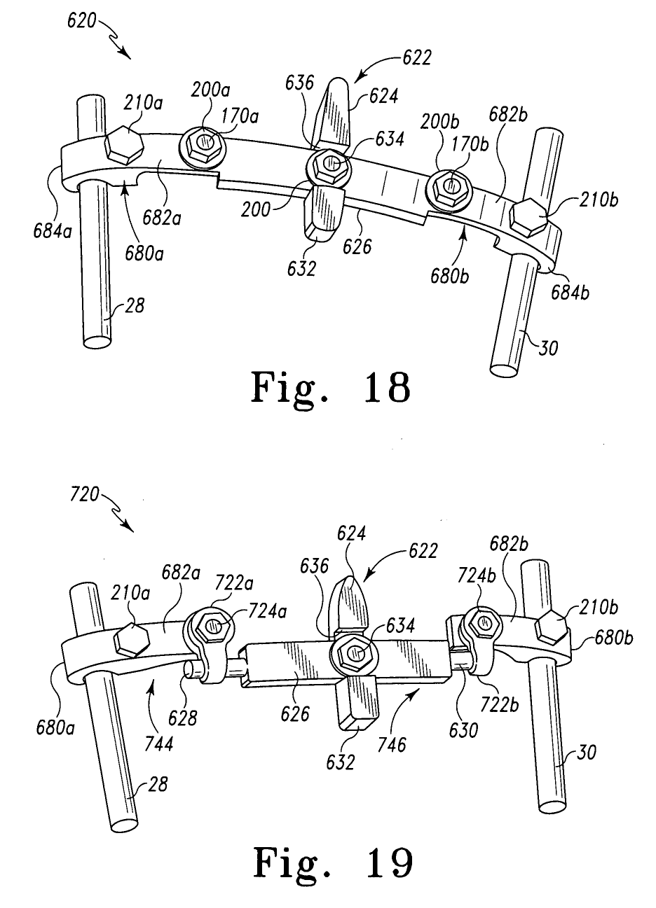

Referring to FIG. 17, there is shown another embodiment of posterior stabilization system 20 designated as stabilization system 520. Stabilization system 520 includes another embodiment replacement body 522 between elongate elements 28, 30. Replacement body 522 includes a spinous process portion 524 extending superiorly from and projecting posteriorly from a central mounting portion 534. Central mounting portion 534 is similar to mounting portion 426 discussed above. Replacement body 522 includes a lamina portion 530 that forms a plate-like portion along the anterior side of spinous process portion 524. An inferiorly extending flange or post 536 extends from mounting portion 534 and oppositely of spinous process portion 524. Post 536 can include a slotted hole or a bore to receive a tether.

First and second connection mechanisms 526, 528 engage replacement body 522 to elongate elements 28, 30 and provide means to permit medial-lateral adjustment of replacement body 522 between elongate elements 28, 30. Mounting portion 534 includes opposite ears 536, 538 with a receiving portion forming a passage to receive the respective arm portion 182a, 182b of linking arms 180a, 180b and a stem 532a, 532b extending from the receiving portion. Arms portions 182a, 182b are located along the posterior or outer surface of mounting portion 534, and are adjustably secured to mounting portion 534 with locking members 200a, 200b engaged to respective ones of the stems 532a, 532b. Replacement body 522 is movable along the linking arms 180a, 180b to locate spinous process portion 524 in the desired location between elongate elements 28, 30.

Referring to FIG. 18, there is shown another embodiment of posterior stabilization system 20 designated as stabilization system 620. Stabilization system 620 includes another embodiment replacement body 622 between elongate elements 28, 30. Replacement body 622 includes a spinous process portion 624 extending superiorly from and projecting posteriorly from a central mounting portion 626. An inferiorly extending flange or post 632 extends from mounting portion 626 and oppositely of spinous process portion 624. Replacement body 622 further includes a recessed portion 636 between spinous process portion 624 and post 632, and includes a stem 634 to which mounting portion 626 is removably mounted. Locking element 200 locks spinous process portion 624 to central mounting portion 626 to coupling mounting portion 626 to recessed portion 636 and between spinous process portion 624 and post 632. In one embodiment, mounting portion 626 includes a central medially-laterally extending slot to provide a connection means to permit medial-lateral adjustment of the location of spinous process portion 624 along mounting portion 626.

Mounting portion 626 extends to opposite laterally extending posts 628, 630 (see FIG. 19) received in the passage of the respective coupling members 170a, 170b. Linking arms 680a, 680b are provided with linking portions 684a, 684b having hook-shaped elements to receive the respective elongate element 28, 30 and a bore to receive respective ones of the securing members 210a, 210b to secure the elongate elements 28, 30 in the hook-shaped elements of linking portions 684a, 684b. Linking arms 680a, 680b also each include a medially extending arm portion 682a, 682b with a hole in the end thereof through which the stem of coupling members 170a, 170b is positioned for engagement with respective ones of the locking members 200a, 200b to engage mounting portion 626 to linking arms 680a, 680b.

Referring to FIG. 19, there is shown another embodiment of posterior stabilization system 20 designated as stabilization system 720. Stabilization system 720 includes replacement body 622 between elongate elements 28, 30 like system 620 in FIG. 18. However, another embodiment connection mechanism 744, 746 is included to provide means to adjustably secure posts 628, 630 of mounting portion 626 to linking arms 680a, 680b. Connection mechanisms 744, 746 include clamps 722a, 722b to adjustably link and secure mounting portion 626 to linking arms 680a, 680b. Clamps 722a, 722b include a C-shaped arrangement with a passage to adjustably receive the respective post 628, 630. The ends of the arms of the C-shaped claim include a hole to receive locking assemblies 724a, 724b to secure the C-shaped clamps around the post 628, 630 and to the respective linking arms 680a, 680b. Locking assemblies 724a, 724b include a stem portion extending through the respective arm portion 682a, 682b of the linking arms 680a, 680b and a locking member like locking member 200 that engages the stem to clampingly engage the adjacent clamp to the respective linking arm.

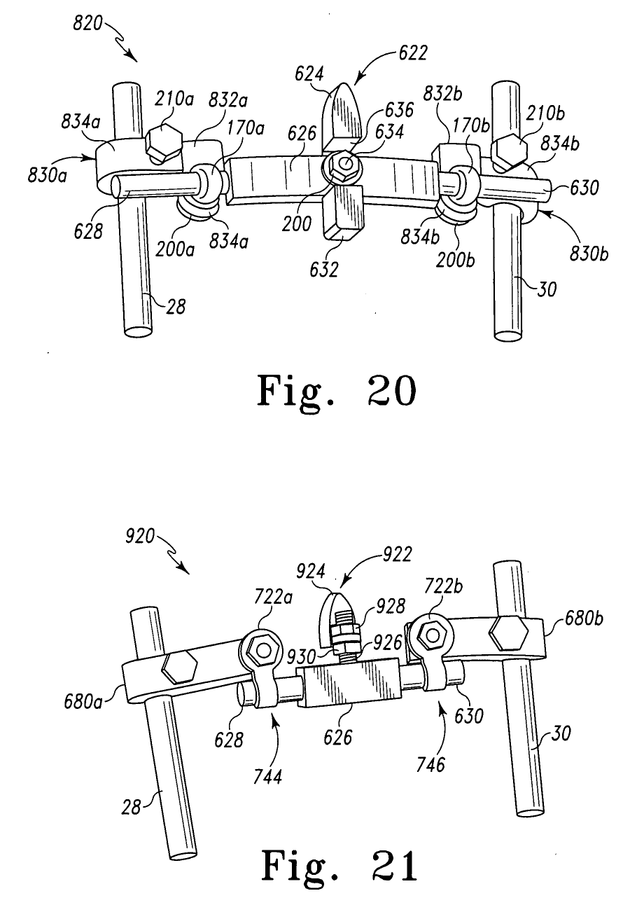

Referring to FIG. 20, there is shown another embodiment of posterior stabilization system 20 designated as stabilization system 820. Stabilization system 820 includes replacement body 622 between elongate elements 28, 30 like system 620 in FIG. 18. However, another embodiment linking arms 830a, 830b are provided that are engaged between elongate elements 28, 30 and mounting portion 626. Linking arms 830a, 830b include ear portions 832a, 832b, respectively, that each defines a hole therethrough offset inferiorly from hook-shaped elements 834a, 834b. Coupling members 170a, 170b extend through the respective ear holes to receive a respective one of the posts 628, 630 through the receiving portion thereof while permitting adjustment in the medial-lateral positioning of the posts 628, 630 and thus replacement body 622 between elongate elements 28, 30. Locking members 200a, 200b are engaged to the coupling members 170a, 170b on the anterior side of replacement body 622 to lock the posts 628, 630 in position therein.

Referring to FIG. 21, there is shown another embodiment of posterior stabilization system 20 designated as stabilization system 920. Stabilization system 920 includes another embodiment replacement body 922 between elongate elements 28, 30. Stabilization system 920 also includes connection mechanisms 744, 746 extending between the elongate elements 28, 30 and replacement body 922 like the connection mechanisms 744, 746 of system 720 in FIG. 19. However, replacement body 922 includes another embodiment spinous process portion 924 extending from mounting portion 626. Replacement body 922 includes a threaded stem 926 extending superiorly from mounting portion 626, and a removable and adjustable spinous process portion 924 adjustably mounted along stem 926. Spinous process portion 924 includes an eyelet 928 that is positioned around stem 926 and threadingly engages stem 926 to allow adjustment in the superior-inferior location of spinous process portion 924. Stem 926 also includes a stop member 930 engaged thereto that is adjustable inferiorly and superiorly to abut eyelet 928 and limit its movement along stem 926.

Spinous process portion 924 includes a superiorly tapered body that is configured to engage an adjacent end of an interpsinous implant. Stabilization system 920 thus permits adjustment of the spinous process portion 924 superiorly and inferiorly relative to mounting portion 626, while connection mechanisms 744, 746 permit medial-lateral adjustability of spinous process portion 924 between elongate elements 28, 30. In one embodiment, multiple spinous process portions 924 of various sizes and shapes are provided in a kit. The desired spinous process portion is selected from the kit and engaged to stem 926 based on criteria determined during the surgery or during pre-operative planning. Other embodiments contemplate other removable connection arrangements for engaging spinous process portion 924 to mounting portion 626.

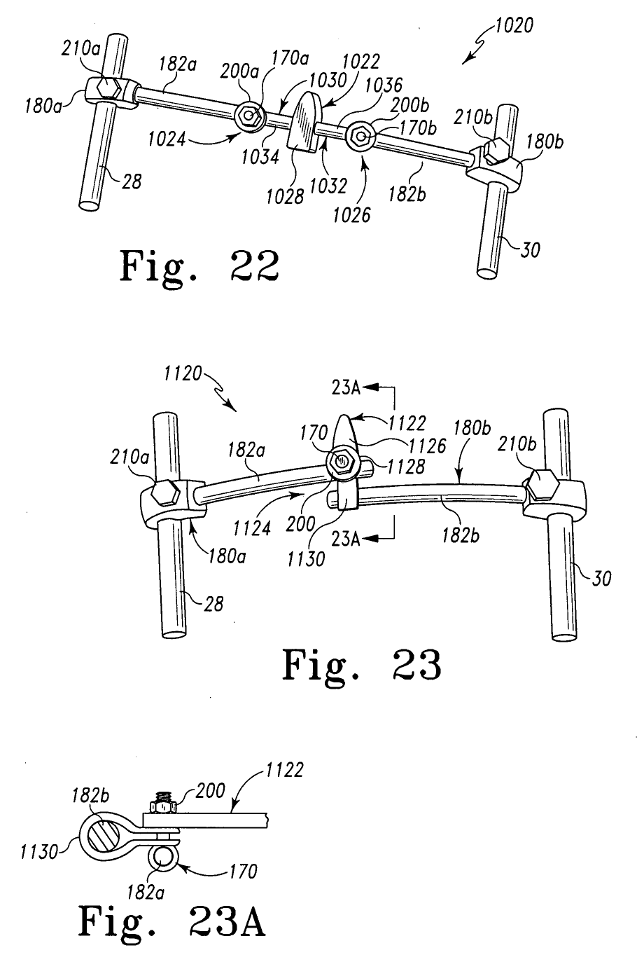

Referring to FIG. 22, there is shown another embodiment of posterior stabilization system 20 designated as stabilization system 1020. Stabilization system 1020 includes another embodiment replacement body 1022 positioned between elongate elements 28, 30 that is secured to linking arms 180a, 180b with adjustable connection mechanisms 1024, 1026. Replacement body 1022 includes a central spinous process portion 1028 and oppositely extending mounting portions 1030, 1032 extending from spinous process portion 1028. Mounting portions 1030, 1032 include an elongated arm portion 1034, 1036, respectively, with an ear portion on the outer end thereof like ear portions 136, 138 discussed above with respect to replacement body 122. Coupling members 170a, 170b receive the arm portion 182a, 182b of the adjacent linking arm 180a, 180b, and extend through the ear portions of the respective mounting portions 1030, 1032 to receive locking members 200a, 200b and engage the linking arms 180a, 180b to the respective mounting portion 1030, 1032. Replacement body 1022 is adjustable medially-laterally along arm portions 182a, 182b to the desired location relative to the vertebral body.

Referring to FIG. 23, there is shown another embodiment of posterior stabilization system 20 designated as stabilization system 1120. Stabilization system 1120 includes another embodiment replacement body 1122 positioned between elongate elements 28, 30 that is secured to linking arms 180a, 180b with adjustable connection mechanism 1124 that provides means to adjust the medial-lateral location of replacement body 1122 along linking arms 180a, 180b. Replacement body 1122 includes a central spinous process portion 1126 with a central eyelet portion 1128 having a through-hole to receive coupling member 170. Replacement body 1122 also includes an inferior clamping portion 1130 with a passage to receive arm portion 182b of linking arm 180b. As shown in FIG. 23A, arm portion 182a of linking arm 180a extends through the receiver portion of coupling member 170, while clamping portion 1130 is positioned on one of the anterior and posterior sides of central portion 1128 (anterior side in the illustrated embodiment.) Coupling member 170 also extends through aligned holes of the clamping portion 1130. Locking member 200 engages coupling member 170 and secures clamping portion 1130 around linking arm 182b and in engagement with central portion 1128. Locking member 200 also secures arm portion 182a in coupling member 170 along the anterior side of central portion 1128.

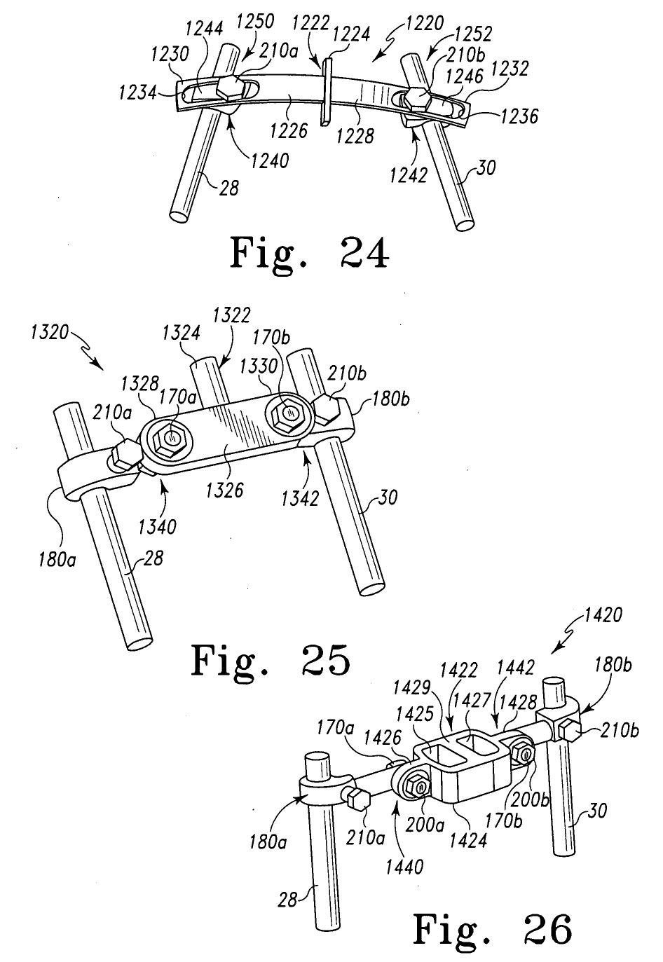

Referring to FIG. 24, there is shown another embodiment of posterior stabilization system 20 designated as stabilization system 1220. Stabilization system 1220 includes another embodiment replacement body 1222 between elongate elements 28, 30. Replacement body 1222 includes a central spinous process portion 1224 that has a plate-like body oriented to extend anteriorly-posteriorly and superiorly-inferiorly. Mounting portions 1226, 1228 extend from opposite sides of replacement body 1222 to outer ear portions 1230, 1232 and are secured to elongate elements 28, 30 with connection mechanisms 1250, 1252. Mounting portions 1226, 1228 each define a plate-like body that extends medially-laterally and superior-inferiorly in a transverse orientation to spinous process portion 1224. Ear portions 1230, 1232 each define a medially-laterally extending slot 1234, 1236, respectively, positioned adjacent to a respective elongate element 28, 30.

Linking arms 1240, 1242 are similar to linking arms 180 discussed above, but do not include an arm portion. Rather, linking arms 1240, 1242 include a hook portion 1244, 1246, respectively, which is secured to the respective elongate element 28, 30 with securing members 210a, 210b, respectively. Securing members 210a, 210b further extend through the adjacent slot 1234, 1236 to couple the respective mounting portion 1226, 1228 to the corresponding linking arm 1240, 1242. Mounting portions 1226, 1228 are adjustable medially-laterally along the slots 1234, 1236 to adjust the medial-lateral positioning of replacement body 1224 between elongate elements 28, 30.

Referring to FIG. 25, there is shown another embodiment of posterior stabilization system 20 designated as stabilization system 1320. Stabilization system 1320 includes another embodiment replacement body 1322 between elongate elements 28, 30. Replacement body 1322 includes a central spinous process portion 1324 that has a rod-like body oriented to extend superiorly from an inferior mounting portion 1326. Mounting portion 1326 has a flat, plate-like body extending medially-laterally to outer ear portions 1328, 1330. Connection mechanisms 1340, 1342 couple ear portions 1328, 1330 to linking arms 180a, 180b to while providing adjustability in the medial-lateral positioning of replacement body 1322 between elongate elements 28, 30. Ear portions 1328, 1330 include through-holes to receive a stem of a respective one of the coupling members 170a, 170b. Linking arms 180a, 180b are coupled to the receiver portion of the respective coupling members 170a, 170b, and when the desired positioning of replacement body 1322 is obtained locking members 200a, 200b secure replacement body 1322 in position between elongate elements 28, 30.

Referring to FIG. 26, there is shown another embodiment of posterior stabilization system 20 designated as stabilization system 1420. Stabilization system 1420 includes a replacement body 1422 between elongate elements 28, 30. Replacement body 1422 includes a central spinous process portion 1424 that has a rectangular body with superiorly-inferiorly extending openings 1425, 1427 and a central strut 1429 extending between anterior and posterior walls of the rectangular body between openings 1425, 1427. Replacement body 1422 also includes a mounting portion including opposite ear portions 1426, 1428 extending from opposite lateral sides of spinous process portion 1424. Ear portions 1426, 1428 are engaged to connection mechanisms 1440, 1442 that permit adjustment in the medial-lateral positioning of replacement body 1422 between elongate elements 28, 30. Ear portions 1426, 1428 each include a through-hole to receive a stem of a respective one of the coupling members 170a, 170b. Linking arms 180a, 180b are coupled to the receiver portion of the respective coupling member 170a, 170b, and when the desired positioning of replacement body 1422 along linking arms 180a, 180b is obtained, locking members 200a, 200b secure replacement body 1422 in position between elongate elements 28, 30.

The components of the systems discussed herein can be made from any suitable biocompatible material. Contemplated materials include metals and metal alloys, polymers, ceramics, elastomers, bone, carbon fiber, and PEEK, for example. The material can be homogenous or composite, and different portions of the implants can be made from different materials to provide desired performance characteristics.

While the invention has been illustrated and described in detail in the drawings and foregoing description, the same is to be considered as illustrative and not restrictive in character. All changes and modifications that come within the spirit of the invention are desired to be protected.

What is claimed is:

1. A posterior spinal stabilization system, comprising:
first and second elongate elements positionable along a spinal column;
a replacement body positionable between said first and second elongate elements, wherein said replacement body includes a spinous process portion alignable along a vertebral body to replace a spinous process; and
connection means for adjustably engaging said replacement body and said first and second elongate elements to one another, wherein said connection means is structured to permit said replacement body to be adjusted between said first and second elongate elements by moving said replacement body along said connection means transversely to said first and second elongate elements.

2. The system of claim 1, further comprising an interspinous spacer engageable to said spinous process portion and being configured to extend from said spinous process portion to a spinous process of a vertebra adjacent to the vertebral body.

3. The system of claim 1, wherein said connection means includes:
first and second linking arms engageable to respective ones of said first and second elongate elements; and
first and second coupling members to engage respective ones of said first and second linking arms to said replacement body.

4. The system of claim 3, further comprising first and second securing members to engage respective ones of said first and second elongate elements to said respective linking arm.

5. The system of claim 4, wherein said replacement body includes a mounting portion extending from said spinous process portion, said mounting portion including opposite ear portions and said first and second coupling members engage said respective one of said first and second linking arms to respective ones of said opposite ear portions.

6. The system of claim 5, wherein each of said ear portions includes a hole and said first and second coupling members are received in said hole of said respective ear portion.

7. The system of claim 1, wherein said first and second elongate elements are elongated spinal rods having a length sized to extend between at least two vertebrae.

8. The system of claim 1, wherein said connection means includes first and second linking arms engaged to said replacement body and to respective ones of said first and second elongate elements, said first and second linking arms each including a hook-shaped end portion forming a receptacle for receiving said respective elongate element and further comprising a securing member securing said respective elongate element against an inner surface of said hook-shaped end portion.

9. The system of claim 8, wherein said first and second linking arms each include a bore in communication with said receptacle thereof and said securing members are engaged in respective ones of said bores in contact with said respective elongate element to secure said respective elongate element in said receptacle.

10. The system of claim 9, wherein said receptacles open away from said replacement body to receive said respective elongate element therein.

11. The system of claim 9, wherein said receptacles open toward said replacement body to receive said respective elongate element therein.

12. The system of claim 8, wherein each of said first and second linking arms includes an ear portion with a hole extending therethrough opposite said hook-shaped end portion thereof; and further comprising a coupling member in each of said holes receiving a respective one of first and second rod portions of said replacement body therein and first and second locking members engageable to respective ones of said coupling members to engage said respective rod portion of said replacement body to said respective linking arm.

13. The system of claim 1, wherein said connection means includes:
first and second linking arms engaged to respective ones of said first and second elongate elements, wherein said replacement body is movable along said first and second linking arms to a desired location between said first and second elongate elements.

14. The system of claim 13, wherein said connection means further includes:
first and second coupling members coupling respective ones of said first and second linking arms to said replacement body.

15. The system of claim 14, wherein said connection means further includes:
first and second locking members engaging respective ones of said first and second coupling members for securing said replacement body in said desired location along said first and second linking arms.

16. The system of claim 13, wherein said connection means includes:
a mounting portion extending from said spinous process portion to each of said elongate elements, said mounting portion including a slot at each end thereof along respective ones of said first and second linking arms; and
first and second securing members extending through respective ones of said slots and engaging said respective linking arm, wherein said mounting portion is movable along said first and second securing members and said first and second securing members are operable to engage said mounting portion to said first and second linking arms in said desired location.

17. The system of claim 16, wherein said first and second securing members further secure respective ones of said first and second elongate elements in said respective linking arm.

18. The system of claim 1, wherein said replacement body includes a mounting portion and said spinous process portion is removably engaged to said mounting portion.

19. The system of claim 18, wherein said mounting portion includes a stem extending superiorly therefrom and said spinous process portion is threadingly engaged about said stem.

20. The system of claim 18, wherein said spinous process portion includes a superiorly extending portion and an inferiorly extending portion with a recess therebetween, said mounting portion being engaged to said spinous process portion in said recess.

21. The system of claim 1, wherein said spinous process portion includes a width in a direction between said first and second elongate elements and said width tapers in a first direction.

22. The system of claim 1, wherein said spinous process portion includes a rectangular body and first and second through-holes extending through said rectangular body, said first and second through-holes oriented in the direction in which said first and second elongate elements extend.

23. The system of claim 1, wherein said replacement body includes a flange portion extending in a direction opposite said spinous process portion, said flange portion including a slotted hole opening therein for receiving a tether.

24. The system of claim 23, wherein said replacement body includes a mounting portion with first and second ear portions extending in opposite directions from one another and transversely to said flange portion.

25. The system of claim 24, wherein said replacement body includes a mounting portion extending transversely to said spinous process portion and said connection means is engaged to said mounting portion, wherein said spinous process portion projects posteriorly from said mounting portion and tapers in width away from mounting portion.

26. The system of claim 1, wherein said spinous process portion extends between a superior end and an inferior end and further comprising a notch for receiving a distraction instrument in at least one of said superior and inferior ends of said spinous process portion.

27. The system of claim 1, wherein said spinous process portion includes a hole for receiving a tether.

28. The system of claim 1, wherein said replacement body includes a lamina portion extending along an anterior side of said spinous process portion, wherein said lamina portion extending outwardly from opposite sides of said spinous process portion.

29. A posterior spinal stabilization system, comprising:
first and second elongate elements;
a replacement body positionable between said first and second elongate elements, wherein said replacement body includes a spinous process portion alignable along a vertebral body to replace a spinous process of the vertebral body; and
first and second linking arms engaged to respective ones of said first and second elongate elements, wherein said replacement body is adjustably connected to said first and second linking arms and is movable along said linking arms to a desired location between said first and second elongate elements.

30. The system of claim 29, further comprising first and second coupling members receiving respective ones of said first and second linking arms and engaged to said replacement body, wherein said coupling members are slidable along said linking arms to move said replacement body to said desired location.

31. The system of claim 30, further comprising first and second locking members engageable to respective ones of said first and second coupling members to lock said replacement body in said desired location along said first and second linking arms.

32. The system of claim 31, further comprising first and second securing members engaging respective ones of said first and second linking arms to secure respective ones of said first and second elongate elements in a receptacle of said respective linking arm.

33. The system of claim 29, wherein said first and second linking arms extend transversely to said elongate elements and said spinous process portion is oriented transversely to said first and second linking arms.

34. The system of claim 33, wherein said spinous process portion includes a body having a width in a direction extending between said first and second elongate elements and said width tapers away from said first and second linking arms.

35. The system of claim 29, wherein said replacement body includes a mounting portion extending transversely to said spinous process portion, and said linking arms are coupled to said mounting portion.

36. The system of claim 35, wherein said replacement body includes a flange portion extending from said spinous process portion and across said mounting portion, said mounting portion extending in opposite directions from said flange portion.

37. The system of claim 36, wherein said spinous process portion and said flange portion protrude posteriorly from said mounting portion.

38. The system of claim 37, wherein said flange portion includes a slotted hole extending therein, said slotted hole opening in a direction opposite said spinous process portion.

39. The system of claim 37, wherein said mounting portion includes an ear portion at opposite ends thereof, said ear portions each including a hole extending therethrough, and further comprising first and second coupling members in each of said holes, said coupling members each including a receiving portion for receiving a respective one of said first and second linking arms.

40. The system of claim 29, wherein at least one of said first and second linking arms includes a rotational stop to contact said replacement body and prevent said replacement body from rotating about said first and second linking arms.

41. The system of claim 40, wherein said rotational stop is formed at an end portion of said at least one linking arm and abuttingly engages an anterior surface of said replacement body to prevent said replacement body from rotating about said at least one linking arm.

42. The system of claim 29, wherein said at least one linking arm is engaged to said replacement body with a coupling member and said at least one linking arm and said coupling member include a keyed arrangement to prevent said replacement body from rotating about said at least one linking arm.

43. A method for posterior spinal stabilization, comprising:
engaging first and second elongate elements posteriorly along a spinal column, the first and second elongate elements being located on opposite sides of a sagittal plane of the spinal column;
positioning a spinous process replacement body between the first and second elongate elements adjacent a vertebra of the spinal column;
securing first and second linking arms to respective ones of the first and second elongate elements;
adjusting a position of the spinous process replacement body along the vertebra in the medial-lateral direction; and
engaging the spinous process replacement body to the first and second linking arms to fix the spinous process replacement body in the position.

44. The method of claim 43, further comprising engaging an interspinous spacer to the spinous process replacement body and a spinous process of an adjacent vertebra.

45. The method of claim 44, wherein the spinous process is located superiorly of the spinous process replacement body.

46. The method of claim 44, further comprising tethering the interspinous spacer to the spinous process replacement body.

47. The method of claim 43, further comprising positioning an interbody device in a disc space along the spinal column.

48. The method of claim 43, wherein the first and second elongate elements are non-parallel to one another along the spinal column.

49. The method of claim 43, further comprising removing at least a portion of a spinous process of the vertebra before positioning the spinous process replacement body between the first and second elongate elements adjacent the vertebra.
